(19) United States
(12) Patent Application Publication
(10) Pub. No.: US 2026/0248730 A1
(43) Pub. Date: Aug. 27, 2026

(54) HARD-SHELL CAPSULE WITH AT LEAST ONE COATING LAYER WITH A LOW OXYGEN PERMEABILITY

(71) Applicant: Evonik Operations GmbH, Essen (DE)

(72) Inventors: Manfred AßMUS, Bickenbach (DE); Kathrin NOLLENBERGER, Darmstadt (DE); Bettina HÖLZER, Bensheim (DE); Hans BÄR, Michelstadt (DE)

(73) Assignee: Evonik Operations GmbH, Essen (DE)

(21) Appl. No.: 18/726,954

(22) PCT Filed: Feb. 20, 2023
(86) PCT No.: PCT/EP2023/054164

§ 371 Date: Jul. 5, 2024

(30) Foreign Application Priority Data
Feb. 22, 2022 (EP) 22157981.6

(51) Int. Cl. A61K 9/48; A61K 47/32

(52) U.S. Cl. CPC A61K 9/4891; A61K 9/4833; A61K 47/32

(57) ABSTRACT

The present invention refers to a precoated hard-shell capsule with at least one coating layer, suitable as container with an improved protective effect against atmospheric oxygen for pharmaceutical or nutraceutical biologically active ingredients and to a method for preparing a pharmaceutical delivering system comprising a hard-shell capsule and a fill, the fill comprising a pharmaceutically or nutraceutically active ingredient.

### FIELD OF THE INVENTION

The present invention refers to a precoated hard-shell capsule having an improved protective effect against atmospheric oxygen with at least one coating layer, suitable as container for pharmaceutical or nutraceutical biologically active ingredients and to a method for preparing a pharmaceutical delivering system comprising such a hard-shell capsule and a fill, the fill comprising at least one pharmaceutically or nutraceutically active ingredient.

### BACKGROUND

Hard-shell capsules are usually made from gelatin or hydroxypropyl methylcellulose (HPMC). Capsules made of these materials dissolve relatively quickly in the gastrointestinal tract. One of the main tasks of capsules is to protect their contents from external influences. However, with regard to atmospheric oxygen, the protective effect still is inadequate.

Polymer-coated hard-shell capsules in the field of pharmaceutical or nutraceutical products are for example disclosed in WO 2019/096833 A1, WO 2020/229178 A1, and WO 2020/229192 A1.

It has been found that even thin film coatings with a particular composition comprising methyl methacrylate (co-) polymers can significantly improve the protective effect against atmospheric oxygen. Applying this film coating to unfilled capsules in a pre-locked state leads to a universally applicable protective container for a wide variety of fillings.

### SUMMARY OF THE INVENTION

The present invention concerns a precoated hard-shell capsule with at least one coating layer, suitable as container for pharmaceutical or nutraceutical biologically active ingredients, wherein the at least one coating layer comprises
i) at least one (meth)acrylate copolymer with a weight average molar mass from 25,000 to 900,000 g/mol and having a glass transition temperature (T_{gm}) from 0 to 200° C., wherein the (meth)acrylate copolymer comprises maximally 50% by weight of methacrylic acid monomer units,
ii) at least one plasticizer with a solubility in water of at least 50 g/l in an amount of maximally 50% by weight relative to the amount of the (meth)acrylate copolymer,
iii) at least one emulsifier with a melting point higher than 40° C. and having a hydrophilic/lipophilic balance (HLB) of 3 to 6,
iv) at least one emulsifier with a melting point lower than 40° C. and having a hydrophilic/lipophilic balance (HLB) of 8 to 16,
and wherein the at least one coating layer has an oxygen permeability of at most 2,500 ml/(m²·d·atm) measured at a thickness of the least one coating layer of 100 μm.

The weight average molar mass is determined by the method according to Adler et al. (Martina Adler, Harald Pasch, Christian Meier, Raimund Senger, Hans-Günter Koban, Michael Augenstein, Günter Reinhold; “Molar mass characterization of hydrophilic copolymers. 1. Size exclusion chromatography of neutral and anionic (meth)acrylate copolymers.”, e-Polymers 2004, no. 055 and Martina Adler, Harald Pasch, Christian Meier, Raimund Senger, Hans-Günter Koban, Michael Augenstein, Günter Reinhold; “Molar mass characterization of hydrophilic copolymers, 2. Size exclusion chromatography of cationic (meth)acrylate copolymers.”, e-Polymers 2005, no. 057).

The glass transition temperature T_{gm} is determined by Differential Scanning calorimetry (DSC) according to ISO 11357-2:2013-05. The determination is performed with a heating rate of 20 K/min. The glass transition temperature T_{gm} is determined by half step height method as described in section 10.1.2 of DIN EN ISO 11357-2:2013-05.

The hydrophilic/lipophilic balance (HLB) is determined by the method according to Griffin, William C. (1954) (“Calculation of HLB Values of Non-Ionic Surfactants” (PDF), Journal of the Society of Cosmetic Chemists, 5 (4): 249-56).

Preferably, the at least one emulsifier with a melting point higher than 40° C. and having a hydrophilic/lipophilic balance (HLB) of 3 to 6 comprised in the at least one coating layer is present in an amount of maximally 25% by weight relative to the amount of the (meth)acrylate copolymer and wherein and the at least one emulsifier with a melting point lower than 40° C. and having a hydrophilic/lipophilic balance (HLB) of 8 to 16 comprised in the at least one coating layer is present in an amount of maximally 10% by weight relative to the amount of the (meth)acrylate copolymer.

The base material of the hard-shell capsule is preferably selected from hydroxypropyl methyl cellulose, starch, gelatine, pullulan and a copolymer of a C₁- to C₄-alkylester of (meth)acrylic acid and (meth)acrylic acid.

Preferably, the at least one coating layer further comprises at least one glidant which is present in an amount of 3 to 75% by weight, based on the total weight of the at least one polymer. The at least one glidant is preferably selected from silica, ground silica, fumed silica, kaolin calcium silicate, magnesium silicate, colloidal silicone dioxide, talc, stearate salts, sodium stearyl fumarate, starch and stearic acid or mixtures thereof.

In a further embodiment, the at least one (meth)acrylate copolymer comprised in the at least one coating layer has a glass transition temperature (T_{gm}) from 40 to 160° C.

The at least one plasticizer of the precoated hard-shell capsule according to the present invention is preferably present in an amount of 2 to 40% by weight, based on the total weight of the at least one (meth)acrylate copolymer.

The at least one plasticizer comprised in the at least one coating layer of the precoated hard-shell capsule according to the present invention is selected from alkyl citrates, alkyl phthalates, diethyl sebacate, polyethylene glycols, propylene glycols or combinations thereof.

The at least one coating layer of precoated hard-shell capsule is preferably present in an amount of 0.1 to 5.8 mg/cm².

The precoated hard-shell capsule according to the present invention preferably comprises a body and a cap, wherein in the closed state the cap overlaps the body either in a pre-locked state or in a final-locked state.

Precoated means in the context of the present invention that the hard-shell capsule is provided with a coating layer prior to be filled with a fill comprising a pharmaceutically or nutraceutically active ingredient.

The present invention further concerns a method for preparing a pharmaceutical delivering system comprising a hard-shell capsule and a fill, the fill comprising a pharmaceutically or nutraceutically active ingredient, wherein the precoated hard-shell capsule according to the present invention is provided in the pre-locked state to a capsule-filling machine, which performs the steps of separating the body and the cap, filling the body with the fill and re-joining the body and the cap in the final-locked state.

The present invention further concerns a method for protecting a pharmaceutically or nutraceutically active ingredient from atmospheric oxygen in a pharmaceutical delivering system comprising a hard-shell capsule and a fill, the fill comprising a pharmaceutically or nutraceutically active ingredient, wherein the precoated hard-shell capsule according to the present invention as described herein is provided in a pre-locked state to a capsule-filling machine, which performs the steps of separating the body and the cap, filling the body with the fill and re-joining the body and the cap in the final-locked state.

### DETAILED DESCRIPTION OF THE INVENTION

### Hard-Shell Capsules

Hard-shell capsules for pharmaceutical or nutraceutical purposes are well known to a skilled person. A hard-shell capsule is a two-piece encapsulation capsule comprising of the two capsule halves, called the body and the cap. The capsule body and cap material usually are made from a hard and sometimes brittle material. The hard-shell capsule comprises a body and a cap. Body and cap are usually of a one end open cylindrical form with closed rounded hemispherical ends on the opposite end. The shape and size of the cap and body are such that the body can be pushed telescopically with its open end into the open end of the cap.

The body and the cap comprise a potential overlapping, matching area (overlap area) outside the body and inside the cap which partially overlap when the capsule is closed in the pre-locked state and totally overlap in the final-locked state. When the cap is partially slid over the overlapping matching area of the body the capsule is in the pre-locked state. When the cap is totally slid over the overlapping matching area of the body the capsule is in the final-locked state. The maintenance of the pre-locked state or of the final-locked state is usually supported by snap-in locking mechanisms of the body and the cap such as matching encircling notches or dimples, preferably elongated dimples.

Usually, the body is longer than the cap. The outside overlapping area of the body can be covered by the cap in order to close or to lock the capsule. In the closed state the cap covers the outside overlap area of the body either in a pre-locked state or in a final-locked state. In the final-locked state the cap covers the outside overlap area of the body in total, in the pre-locked state the cap overlaps the outside overlapping area of the body only partially. The cap can be slid over the body to be fixed in usually one of two different positions in which the capsule is closed either in a pre-locked state or in a final-locked state.

Hard-shell capsules are commercially available in different sizes. Hard-shell capsules are usually delivered as empty containers with the body and cap already positioned in the pre-locked state and on demand as separate capsules halves, bodies and caps. The pre-locked hard-shell capsules can be provided to a capsule-filling machine, which performs the opening, filling and closing of the capsule into the final-locked state. Usually, hard-shell capsules are filled with dry materials, for instance with powders or granules, or viscous liquids comprising a biologically active ingredient.

The cap and body are provided with closure means that are advantageous for the pre-locking (temporary) and/or final locking of the capsule. Therefore, elevated points can be provided on the inner wall of the cap and somewhat larger indented points are provided on the outer wall of the body, which are arranged so that when the capsule is closed the elevations fit into the indentations.

Alternatively, the elevations can be formed on the outer wall of the body and the indentations on the inner wall of the cap. Arrangements in which the elevations or indentations arranged in a ring or spiral around the wall. Instead of the point-like configuration of the elevations and indentations, these may encircle the wall of the cap or body in an annular configuration, although advantageously recesses and openings are provided which enable an exchange of gases into and out of the capsule interior. One or more elevations can be provided in an annular arrangement around the inner wall of the cap and the outer wall of the body such that, in the final-locked position of the capsule, an elevation on the cap is located adjacent to an elevation on the body. Sometimes elevations are formed on the outside of the body close to the open end and indentations are formed in the cap close to the open end such that the elevations on the body latch into the indentations in the cap in the final-locked position of the capsule. The elevations can be such that the cap can be opened in the pre-locked state at any time without damage to the capsule or, alternatively, so that once it has been closed the capsule cannot be opened again without destroying it. Capsules with one or more such latching mechanisms (latches) (for example two encircling grooves) are preferred. More preferred are capsules with at least two such latching means which secure the two capsule parts to different degrees. In a part of this kind, a first latching (dimples or encircling notches) means can be formed close to the openings in the capsule cap and the capsule body and a second latching (encircling notches) can be shifted somewhat further towards the closed end of the capsule parts. The first latching means secure the two capsule parts less strongly than the second does. This variant has the advantage that after the production of the empty capsules the capsule cap and capsule body can initially be pre-locked joined together using the first latching mechanism. In order to fill the capsule, the two capsule parts are then separated again. After filling, the two capsule parts are pushed together until the second set of latches firmly secures the capsule parts in a final-locked state.

Preferably, the body and the cap of the hard-shell capsule are comprising each encircling notches and/or dimples in the area, where the cap can be slid over the body. Encircling notches of the body and dimples of the cap match to each other to provide a snap-in or snap into-place mechanism. The dimples can be circular or elongated (oval) in the longitudinal direction. Encircling notches of the body and encircling notches of the cap (closely matched rings) also match to each other to provide a snap-in or snap into-place mechanism. This allows the capsule to be closed by a snap-into-place mechanism either in a pre-locked state or in a final-locked state.

Preferably, matching encircling notches of the body and elongated dimples of the cap are used to fix the body and the cap to each other in the pre-locked state. Matching encircling notches of the body and the cap are preferably used to fix or lock the body and the cap to each other in the final-locked state.

The area, where the cap can be slid over the body can be called the overlapping area of the body and the cap or briefly the overlap area. If the cap overlaps the body only partially, maybe to 20 to 90 or 60 to 85% of the overlap area, the hard-shell capsule is only partially closed (pre-locked). Preferably, in the presence of a locking mechanism, like matching encircling notches and/or dimples in body and cap, the partially closed capsule can be called pre-locked. When the capsule is polymer-coated in the pre-locked state the coating will cover the completely outer surface including that part of the overlap area of the body and cap that is not overlapped by the cap in this pre-locked state. When the capsule is polymer-coated in the pre-locked state and then closed to the final-locked state the coating of that part of the overlap area of the body and cap that was not overlapped by the cap in the pre-locked state will then become covered by the cap. The presence of that part of the coating which is then enclosed in the final-locked state between the body and the cap is sufficient for the hard-shell capsule to be tightly sealed.

If the cap overlaps the body the total overlapping area of the body, the hard-shell capsule is finally closed or in the final-locked state. Preferably, in the presence of a locking mechanism, like matching encircling notches and/or dimples in body and cap, the finally closed capsule can be called final-locked.

Usually, dimples are preferred for the fixing the body and the cap in the pre-locked state. As a non-binding rule the matching area of dimples is smaller than the matching area of encircling notches. Thus snapped-in dimples can be snapped-out again by applying less forces than those that would be necessary to snap-out a snapped-in fixation by matching encircling notches.

The dimples of the body and cap are located in the area, where the cap can be slid over the body match to each other in the pre-locked state by a snap in or snap into-place mechanism. There can be for example 2, 4, or preferably 6 notches or dimples located distributed circular around the cap.

Usually, the dimples of the cap are and the encircling notches of the body in the area, where the cap can be slid over the body match to each other so that they that allow the capsule to be closed by a snap-into-place mechanism in the pre-locked state. In the pre-locked state, the hard-shell capsule can be re-opened manually or by a machine without damaging, because the forces needed to open are comparatively low. Thus, the “pre-locked state” is sometimes designated also as “loosely capped”.

Usually, the encircling notches or matching locking rings of the body and the cap in the area, where the cap can be slid over the body match to each other so that they that allow the capsule to be closed by a snap-into-place mechanism in the final-locked state. In the final-locked state, the hard-shell capsule cannot or can only be hardly re-opened manually or by a machine without damaging, because the forces needed to open are comparatively high.

Usually dimples and the encircling notches are formed in the capsule body or capsule cap. When the capsule parts provided with these elevations and indentations are fitted into one another, ideally defined uniform gaps of from 10 microns to 150 microns, more particularly 20 microns to 100 microns, are formed along the contact surface between the capsule body and the capsule cap placed thereon.

Preferably, the body of the hard-shell capsule comprises a tapered rim. The tapered rim prevents the rims of the body and the cap to collide and becoming damaged when the capsule is closed manually or by a machine.

In contrast to a hard-shell capsule, a soft-shell capsule is a welded one-piece encapsulation capsule. A soft gel capsule is often made from blow molded soft gelling substances and is usually filled with liquids comprising a biologically active ingredient by injection. The invention is not concerned with welded soft-shell one-piece encapsulation capsules.

### Sizes of Hard-Shell Capsules

A closed, final-locked hard-shell capsule can have a total length in the range from about 5 to 40 mm. The diameter of the cap can be in the range from about 1.3 to 12 mm. The diameter of the body can be in the range from about 1.2 to 11 mm. The length of the cap can be in the range from about 4 to 20 mm and that of the body in the range from 8 to 30 mm. The fill volume can be between about from 0.004 to 2 ml. The difference between the pre-locked length and the final-locked length can be about 1 to 5 mm.

Capsules can be divided into standardized sizes for example from sizes 000 to 5. A closed capsule of size 000 has, for example, a total length of about 28 mm with an outer diameter of the cap of about 9.9 mm and an outer diameter of the body of about 9.5 mm. The length of the cap is about 14 mm, that of the body about 22 mm. The fill volume is about 1.4 ml.

### Material of the Body and the Cap

The base material of the body and the cap can be selected from hydroxypropyl methyl cellulose, starch, gelatin, pullulan, and a copolymer of C₁- to C₄-alkylester of (meth)acrylic acid and (meth)acrylic acid. Preferred are hard-shell capsules where body and cap are comprising or consisting of HPMC or gelatin, most preferred is HPMC because of its good adhesion properties for the polymer coating.

### The at Least One Coating Layer

### Polymer or Polymer Mixture Comprised in the at Least One Coating Layer

The at least one (meth)acrylate copolymer comprised in the at least one coating layer is preferably a film-forming polymer and can be selected from the group of anionic (meth)acrylate copolymers, cationic (meth)acrylate copolymers and neutral (meth)acrylate copolymers having a weight average molar mass from 25,000 to 900,000 g/mol and having a glass transition temperature (T_{gm}) from 0 to 200° C. or any mixture thereof, wherein these (meth)acrylate copolymers comprise maximally 50% by weight of methacrylic acid monomer units.

The weight average molar mass is determined by the method according to Adler et al. (Martina Adler, Harald Pasch, Christian Meier, Raimund Senger, Hans-Günter Koban, Michael Augenstein, Günter Reinhold; “Molar mass characterization of hydrophilic copolymers. 1. Size exclusion chromatography of neutral and anionic (meth)acrylate copolymers.”, e-Polymers 2004, no. 055 and Martina Adler, Harald Pasch, Christian Meier, Raimund Senger, Hans-Günter Koban, Michael Augenstein, Günter Reinhold; “Molar mass characterization of hydrophilic copolymers, 2. Size exclusion chromatography of cationic (meth)acrylate copolymers.”, e-Polymers 2005, no. 057).

The glass transition temperature T_{gm} is determined by Differential Scanning calorimetry (DSC) according to ISO 11357-2:2013-05. The determination is performed with a heating rate of 20 K/min. The glass transition temperature T_{gm} is determined by half step height method as described in section 10.1.2 of DIN EN ISO 11357-2:2013-05.

The coating layer may comprise a (meth)acrylate copolymer selected from copolymers comprising 0 to maximally 50% by weight of polymerized units of methacrylic acid and comprising polymerized units of ethyl acrylate, of methacrylic acid and methyl methacrylate, of ethyl acrylate and methyl methacrylate or of methacrylic acid, methyl acrylate and methyl methacrylate, from a mixture of a copolymer comprising polymerized units of methacrylic acid and ethyl acrylate with a copolymer comprising polymerized units of methyl methacrylate and ethyl acrylate and a mixture of a copolymer comprising polymerized units of methacrylic acid, methyl acrylate and methyl methacrylate with a copolymer comprising polymerized units of methyl methacrylate and ethyl acrylate.

The coating layer may comprise a (meth)acrylate copolymer comprising polymerized monomer units of 60 to 65% by weight of methyl methacrylate, 30% by weight of ethyl acrylate and 5 to 10% by weight of 2-(trimethylamonio)ethyl methacrylate.

The coating layer may comprise a (meth)acrylate copolymer comprising polymerized monomer units of 30% by weight of methyl methacrylate and 70% by weight of ethyl acrylate.

The coating layer may comprise a (meth)acrylate copolymer comprising polymerized monomer units of 40 to 50% by weight of methacrylic acid and 50 to 60% by weight of ethyl acrylate. Suitable is e. g. a copolymer comprising polymerized monomer units of 50% by weight of methacrylic acid and 50% by weight of ethyl acrylate.

The coating layer may comprise a (meth)acrylate copolymer comprising polymerized units of 5 to 15% by weight methacrylic acid, 60 to 70% by weight of methyl acrylate and 20 to 30% by weight methyl methacrylate. A suitable copolymer may be a copolymer polymerized from 25% by weight of methyl methacrylate, 65% by weight of methyl acrylate and 10% by weight of methacrylic acid.

The coating layer may comprise a (meth)acrylate copolymer comprising polymerized monomer units of 60 to 80% of ethyl acrylate and 40 to 20% by weight of methyl methacrylate.

The coating layer may comprise a (meth)acrylate copolymer comprising polymerized units of 40 to 50% by weight of methacrylic acid and 60 to 50% by weight of methyl methacrylate, preferably a copolymer polymerized from 50% by weight of methyl methacrylate and 50% by weight of methacrylic acid.

The coating layer may comprise a (meth)acrylate copolymer comprising polymerized monomer units of 20 to 40% by weight of methacrylic acid and 60 to 80% by weight of methyl methacrylate, preferably a copolymer polymerized from 70% by weight methyl methacrylate and 30% by weight methacrylic acid.

The coating layer may also comprise both, a (meth)acrylate copolymer comprising polymerized monomer units of 30% by weight of methyl methacrylate and 70% by weight of ethyl acrylate and a (meth)acrylate copolymer comprising polymerized monomer units of 50% by weight of ethyl acrylate and 50% by weight of methacrylic acid.

The coating layer, which can be a single layer or can comprise or consist of two or more individual layers, can comprise in total 10 to 100, 20 to 95, 30 to 90% by weight of one or more polymers, preferably (meth)acrylate copolymer(s). The proportions of monomers mentioned for the respective polymers in general add up to 100% by weight.

### Plasticizers

The at least one coating layer of the hard-shell capsule comprises maximally 50% by weight relative to the amount of the (meth)acrylate copolymer of at least one plasticizer (e.g. derived from fatty acids) with a solubility in water at least 50 g/l.

Plasticizers achieve through physical interaction with a polymer a reduction in the glass transition temperature and promote film formation, depending on the added amount. Suitable substances usually have a molecular weight of between 100 and 20,000 g/mol and comprise one or more hydrophilic groups in the molecule, e. g. hydroxyl, ester or amino groups.

Examples of suitable plasticizers are alkyl citrates, alkyl sebacates, diethyl sebacate, polyethylene glycols, and polypropylene glycols. Preferred plasticizers are triethyl citrate (TEC), acetyl triethyl citrate (ATEC), diethyl sebacate, polyethylene glycols, and polypropylene glycols or mixtures thereof.

Addition of the plasticizers to the formulation can be carried out in a known manner, directly, in aqueous solution or after thermal pre-treatment of the mixture. It is also possible to employ mixtures of plasticizers.

### Emulsifiers

The at least one coating layer of the precoated hard-shell capsule according to the present invention comprises at least one emulsifier with a melting point (Tₘ) higher than 40° C. and having a hydrophilic/lipophilic balance (HLB) of 3 to 6 and at least one emulsifier with a melting point (Tₘ) lower than 40° C. and having a hydrophilic/lipophilic balance (HLB) of 8 to 16. Both types of emulsifiers (Tₘ>40° C. with HLB=3-6 and Tₘ<40° C. with HLB=8-16) preferably are non-ionic surfactants.

The HBL Value can be determined according to Griffin, William C. (1954), “Calculation of HLB Values of Non-Ionic Surfactants” (PDF), Journal of the Society of Cosmetic Chemists, 5 (4): 249-56.

Suitable emulsifiers having a melting point (Tₘ) higher than 40° C. and an HLB of 3 to 6 are for example glycerol monosterate (Tₘ: 56-58° C., HLB: about 3.8) or sorbitan monostearate (Span 60, Tₘ=53-57° C.; HLB=4.7).

Suitable emulsifiers having a melting point (Tₘ) lower than 40° C. and an HLB of 8 to 16 are for example polysorbate 80 (Tₘ: −21° C., HLB: about 12), polyoxyethylene (4) laurylether (Brij® 30, Tₘ=14° C., HLB=9.7), polyoxyethylene (10) oleylether (Brij® 97, Tₘ=10° C., HLB=12.4) or sorbitan monolaurate (Span 20, liquid at 21° C., HLB=8.6.

### Glidants

The at least one coating layer may further comprise at least one glidant which is present in an amount of 3 to 75% by weight, based on the total weight of the at least one polymer. Glidants usually have lipophilic properties. They prevent agglomeration of cores during film formation of the film forming polymers.

The at least one glidant is preferably selected from silica, for example commercially available under the tradenames RXCIPIENTS® GL100 or RXCIPIENTS® GL200, ground silica, fumed silica, kaolin calcium silicate, magnesium silicate, colloidal silicone dioxide, furthermore talc, stearate salts like calcium stearate, magnesium stearate, zinc stearate, sodium stearyl fumarate, starch, stearic acid, preferably talc, magnesium stearate, and colloidal silicon dioxide, or mixtures thereof.

Standard proportions for use of glidants in the inventive coating range between 0.5 and 100% by weight, preferably 3 to 75% by weight, more preferably 5 to 50% by weight, most preferably 5 to 30% by weight, relative to the total weight of the at least one polymer.

### Amount and Thickness of the at Least One Coating Layer

The at least one functional coating layer is present in an amount of 0.1 to 5.8 mg/cm², preferably in an amount of 2.2 to 4.8 mg/cm² to build a closed film on the hard-shell capsule as determined by calculation or determined by scanning electron microscopy (SEM) or micro computed tomography (micro-CT).

For hard-shell capsules, the amount of the coating layer should not be too high. If the amount of coating layer applied is too high this may result in difficulties to process the polymer-coated pre-locked hard-shell capsules subsequently in a capsule-filling machine. If the amount of coating layer is less than 5.8 mg/cm², for instance 2 to 4 mg/cm² or 1 to 5 mg/cm² or 1 to 5.8 mg/cm² usually no problem with standard capsule-filling machines without modification will occur. In the range from 4 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 1 to about 8 mg/cm².

For a hard-shell capsule of size #0, the amount of the coating layer should not be too high. If the amount of coating layer applied is too high this may result in difficulties to process the polymer-coated pre-locked hard-shell capsules subsequently in a capsule-filling machine. If the amount of coating layer is less than 5 mg/cm², for instance 1 to 4 mg/cm² usually no problem with standard capsule-filling machines without modification will occur. In the range from 4 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 1 to about 8 mg/cm².

For a hard-shell capsule of size #1, the amount of the coating layer should not be too high. If the amount of coating layer applied is too high this may result in difficulties to process the polymer-coated pre-locked hard-shell capsules subsequently in a capsule-filling machine. If the amount of coating layer is less than 4 mg/cm², for instance 1 to 3.5 mg/cm² usually no problem with standard capsule-filling machines without modification will occur. In the range from 3.5 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 1 to about 8 mg/cm².

For a hard-shell capsule of size #3, the amount of the coating layer should not be too high. If the amount of coating layer applied is too high this may result in difficulties to process the polymer-coated pre-locked hard-shell capsules subsequently in a capsule-filling machine. If the amount of coating layer is less than 3 mg/cm², for instance 1 to 2.5 mg/cm² usually no problem with standard capsule-filling machines without modification will occur. In the range from 2.5 and up to about 6 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 1 to about 6 mg/cm².

If the amount of coating layer applied is too high there will be also an assembly of too much coating layer at the rim of the cap where the gap between body and cap is in the pre-locked state. This may result after drying in fissures of the coating layer when the coated pre-locked hard-shell capsule is opened manually or in a machine. The fissures may result in a later leakage of the capsule. Finally, a too thick coating may result in difficulties or make it impossible to close the opened coated hard-shell capsule to the final-locked state since the coating layer is thicker than the gap in the overlapping area between the body and the cap.

As a rough rule the coating layer on the hard-shell capsule can be applied in an amount (=a total weight gain) of 0.1 to 10, 0.5 to 9, 1.0 to 8, 1.5 to 5.5, 1.5 to 4 mg/cm².

As a rough rule the coating layer on the hard-shell capsule may have an average thickness of about 5 to 100, 10 to 50, 15 to 75 μm.

As a rough rule the coating layer on the hard-shell capsule can be applied in an amount of 5 to 50, preferably 8-40% dry weight in relation to the weight of the pre-locked capsule.

With this guidance a skilled person will be able to adjust the amounts of the coating layer in a range between too low and too high.

The at least one coating layer has an oxygen permeability of at most 2,500 ml/(m²·d·atm) measured at a thickness of the least one coating layer of 100 μm.

Example of coating layer thickness determination via Scanning Electron Microscopy (SEM) Investigation

In order to perform a coating layer thickness evaluation, the following procedures are applied consisting of a suitable sample size calculation, random sampling of the number of samples, preparation of sample, determination of layer thickness and result evaluation.

### Sample Number Calculation Equation:

$$sample size = \frac{\frac{{z}^{2} \times p ( 1 - p )}{{e}^{2}}}{1 + ( \frac{{z}^{2} \times p ( 1 - p )}{{e}^{2} N} )}$$

N=Batch Size
e=Error Margin
Z=Z-Value representing the confidence level
p=Standard of deviation

| Confidence level | Z-value |
|---|---|
| 80% | 1.28 |
| 85% | 1.44 |
| 90% | 1.65 |
| 95% | 1.96 |
| 99% | 2.58 |

### Example of Calculation

N=457,200 [units]
e=0.1
Z=1.96
p=0.5

$$sample size = \frac{\frac{{1.96}^{2} \times 0.5 ( 1 - 0.5 )}{{0.1}^{2}}}{1 + ( \frac{{1.96}^{2} \times 0.5 ( 1 - 0.5 )}{{0.1}^{2} 457.2} )}$$

$$sample size = 96.06$$

### Analytical Method

SEM Equipment Set-Up
SEM JEOL JSM-IT300 (Scanning Electron Microscope)
Manufacturer: JEOL Ltd.
Technical specifications EM-standard parameter
Adjustable SEM acceleration voltage
theoretical: 200 to 30 000 V (SEM 5 to 10 KV; EDS 15 kV)
Variable flow of electrons from a tungsten filament (cathode)
Vacuum system: Rotary pump/turbo molecular pump
Maximum sample diameter->vacuum chamber door (max.: 20×7×7 cm³)
X-Y-Z-rotation-tilt: totally motorized!
Working distance (WD): 5 to 70 mm (common: 10 mm)
Sample rotation: 360° Sample tilting: −5 to max. 90° (depending on WD) (EDX: −3° to) 3°
Magnification: 10× to 300 000× (20× to 100 000×)
Maximum resolution: ~3 nm
Detectors: Secondary Electrons (SE)
Back Scattered Electrons (BSE, 5 segments)
Energy dispersive X-Ray Analysis (EDS)

### SEM Method Description:

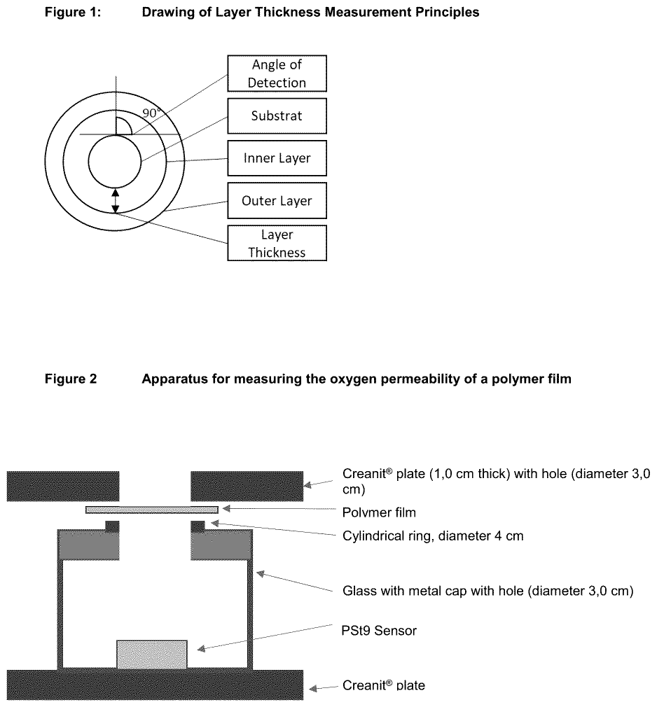

Samples are to be randomly taken from the batch of coated dose units i.e. precoated hard-shell capsules. The taken samples were broken with a sharp-edged cutter, typically into two unit halves, at room temperature. The broken pieces were fixed in an upright position on the sample mounting disc. The samples are to be investigated at an adequate magnification which corresponds to the precoated hard-shell capsule dimensions. The layer thickness will be determined in a 90° angle to the substrate. The single values are note and the mean average and standard deviation is calculated. A drawing of layer thickness measurement principles is provided in FIG. 1.

### Biologically Active Ingredient

The process as disclosed refers to a polymer coated hard-shell capsule, filled with a fill comprising at least one biologically active ingredient. A biologically active ingredient may be defined as an ingredient that may after delivery or intake confers a preventive or therapeutical effect in an animal or human body. The biologically active ingredient is preferably a pharmaceutically active ingredient and/or a nutraceutically active ingredient.

The biologically active ingredient is preferably a pharmaceutical active ingredient and/or a nutraceutical active ingredient and/or a cosmetically active ingredient. Even though it is possible that certain biologically active ingredients are contained in the respective coating layers, it is preferred that the biologically active ingredient is contained in the fill-in. In particular, if the biologically active ingredient is nucleic acid, the biologically active ingredient is only contained in the fill-in.

### Pharmaceutically or Nutraceutically Active Ingredients Comprised in the Pharmaceutical Delivering System

The invention is preferably useful for immediate, delayed release or sustained release formulated pharmaceutical or nutraceutical dosage forms with a fill-in of pharmaceutically or nutraceutically active ingredients.

Suitable therapeutic and chemical classes of pharmaceutically active ingredients which members can be used as fill-in for the described polymer-coated hard-shell capsules are for instance: analgesics, antibiotics or anti-infectives, antibodies, antiepileptics, antigens from plants, antirheumatics, benzimidazole derivatives, beta-blocker, cardiovascular drugs, chemotherapeutics, CNS drugs, digitalis glycosides, gastrointestinal drugs, e.g. proton pump inhibitors, enzymes, hormones, liquid or solid natural extracts, oligonucleotides, peptide, hormones, proteins, therapeutic bacteria, peptides, proteins (metal) salt i.e. aspartates, chlorides, urology drugs, vaccines.

In a preferred embodiment the pharmaceutically active ingredient is a nucleic acid, more preferably a nucleic acid agent can be DNA, RNA, or combinations thereof. In some embodiments, a nucleic acid agent can be an oligonucleotide and/or polynucleotide. In some embodiments, a nucleic acid agent may be an oligonucleotide and/or modified oligonucleotide (including, but not limited to, modifications through phosphorylation); an antisense oligonucleotide and/or modified antisense oligonucleotide (including, but not limited to, modifications through phosphorylation). In some embodiments, a nucleic acid agent can comprise cDNA and/or genomic DNA. In some embodiments, a nucleic acid agent can comprise non-human DNA and/or RNA (e.g., viral, bacterial, or fungal nucleic acid sequences). In some embodiments, a nucleic acid agent can be a plasmid, cosmid, gene fragment, artificial and/or natural chromosome (e.g., a yeast artificial chromosome), and/or a part thereof. In some embodiments, a nucleic acid agent can be a functional RNA (e.g., a mRNA, a tRNA, an rRNA and/or a ribozyme). In some embodiments, a nucleic acid agent can be an RNAi-inducing agent, small interfering RNA (siRNA), short hairpin RNA (shRNA), and/or microRNA (miRNA). In some embodiments, a nucleic acid agent can be a peptide nucleic acid (PNA). In some embodiments, a nucleic acid agent can be a polynucleotide comprising synthetic analogues of nucleic acids, which may be modified or unmodified. In some embodiments, a nucleic acid agent can comprise various structural forms of DNA including single-stranded DNA, double-stranded DNA, supercoiled DNA and/or triple-helical DNA; Z-DNA; and/or combinations thereof. Further suitable nucleic acids are for example disclosed in WO 2012103035 A1, which are incorporated by reference.

Further examples of drugs that can be used as fill-in for the described polymer-coated hard-shell capsules are for instance acamprosat, aescin, amylase, acetylsalicylic acid, adrenalin, 5-amino salicylic acid, aureomycin, bacitracin, balsalazine, beta carotene, bicalutamid, bisacodyl, bromelain, bromelain, budesonide, calcitonin, carbamacipine, carboplatin, cephalosporins, cetrorelix, clarithromycin, chloromycetin, cimetidine, cisapride, cladribine, clorazepate, cromalyn, 1-deaminocysteine-8-D-arginine-vasopressin, deramciclane, detirelix, dexlansoprazole, diclofenac, didanosine, digitoxin and other digitalis glycosides, dihydrostreptomycin, dimethicone, divalproex, drospirenone, duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormon (hGH), ibuprofen, ilaprazole, insulin, Interferon, Interleukin, Intron A, ketoprofen, lansoprazole, leuprolidacetat lipase, lipoic acid, lithium, kinin, memantine, mesalazine, methenamine, milameline, minerals, minoprazole, naproxen, natamycin, nitrofurantion, novobiocin, olsalazine, omeprazole, orothates, pancreatin, pantoprazole, parathyroidhormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, thrypsine, valproic acid, vasopressin, vitamins, zinc, including their salts, derivatives, polymorphs, isomorphs, or any kinds of mixtures or combinations thereof.

It is evident to a skilled person that there is a broad overlap between the terms pharmaceutically and nutraceutically active ingredients, excipients and compositions respectively a pharmaceutical or a nutraceutical dosage form. Many substances listed as nutraceuticals may also be used as pharmaceutically active ingredients. Depending on the specific application and local authority legislation and classification, the same substance can be listed as a pharmaceutically or a nutraceutically active ingredient respectively a pharmaceutical or a nutraceutical composition or even both.

Nutraceuticals are well known to the skilled person. Nutraceuticals are often defined as extracts of foods claimed to have medical effects on human health. Thus, nutraceutically active ingredients may display pharmaceutical activities as well: Examples for nutraceutically active ingredients can be resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Thus, it is clear that many substances listed as nutraceuticals may also be used as pharmaceutically active ingredients. Typical nutraceuticals or nutraceutically active ingredients that can be used as fill-in for the described polymer-coated hard-shell capsules may also include probiotics and prebiotics. Probiotics are living microorganisms believed to support human or animal health when consumed. Prebiotics are nutraceuticals or nutraceutically active ingredients that induce or promote the growth or activity of beneficial microorganisms in the human or animal intestine.

Examples for nutraceuticals are resveratrol from grape products, omega-3-fatty acids or pro-anthocyanines from blueberries as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or antocyanins from berries. Sometimes the expression neutraceuticals or nutriceuticals are used as synonyms for nutraceuticals. Preferred biologically active ingredients are metoprolol, mesalamine and omeprazole.

### Process for Preparing a Coated Hard-Shell Capsule

Described is a process for preparing a polymer-coated hard-shell capsule, suitable as container for biologically active ingredients, wherein the hard-shell capsule comprises a body and a cap, wherein in the closed state the cap overlaps the body either in a pre-locked state or in a final-locked state, wherein the hard-shell capsule is provided in the pre-locked state and spray-coated with a coating solution, suspension or dispersion according to the present invention to create a coating layer which covers the outer surface of the hard-shell capsule in the pre-locked state.

In a further process step the pre-locked hard-shell capsule can be provided with a fill comprising at least one biologically active ingredient and is closed to the final-locked state.

In such a further process step the polymer-coated hard-shell capsule in the pre-locked state can be opened, filled with a fill comprising a biologically active ingredient, and is closed in the final-locked state. This further process step is preferably performed in that the coated hard-shell capsule in the pre-locked state is provided to a capsule-filling machine, which performs the opening, filling with a fill comprising at least one biologically active ingredient and closing of the polymer-coated hard-shell capsule to the final-locked state.

This further process step results in a final-locked polymer-coated hard-shell capsule, which is a container for at least one biologically active ingredient. The final-locked polymer-coated hard-shell capsule, which as a container for at least one biologically active ingredient is a pharmaceutical or nutraceutical dosage form.

The dosage form preferably comprises a polymer-coated hard-shell capsule in the final-locked state containing a fill comprising at least one biologically active ingredient, wherein the polymer-coated hard-shell capsule comprises a coating layer according to the invention, where the coating layer covers the outer surface area of the capsule in the pre-locked state but not the overlapping area where the cap covers the body in the pre-locked state.

A coating suspension comprising the at least one polymer, the at least one glidant and the at least one emulsifier can contain an organic solvent, for instance acetone, iso-propanol or ethanol. The concentration of dry weight material in the organic solvent can be about from 5 to 50% by weight of polymer. A suitable spraying concentration can be about 5 to 25% by dry weight.

A coating suspension can be the dispersion of the at least one polymer, the at least one glidant and the at least one emulsifier in an aqueous medium, for instance water or a mixture of 80% by weight or more of water and 20% or less by weight of water-soluble solvents, such as acetone or isopropanol. A suitable concentration of dry weight material in the aqueous medium can be from about 5 to 50% by weigh. A suitable spraying concentration can be about 5 to 25% by dry weight.

The spray coating is preferably performed by spraying the coating solution or dispersion onto the pre-locked capsules in a drum coater or in a fluidized bed coating equipment.

The surface tension of the coating solution, suspension or dispersion is preferably determined according to the measurement as described in example 1.

### Process for Preparing a Fill for the Dosage Form

Suitable processes for preparing the fill for the pharmaceutical or nutraceutical dosage form are well known to a skilled person. A suitable process for preparing the fill for the at least one pharmaceutical or nutraceutical dosage form as disclosed herein can be by forming a core comprising the biologically active ingredient in the form of pellets by direct compression, compression of dry, wet or sintered granules, by extrusion and subsequent rounding off, by wet or dry granulation, by direct pelleting or by binding powders onto active ingredient-free beads or neutral cores or active ingredient-containing particles or pellets and optionally by applying coating layers in the form of aqueous dispersions or organic solutions in spray processes or by fluidized bed spray granulation.

### Capsule Filling Machine

The polymer-coated hard-shell capsule is provided in the pre-locked state to a capsule-filling machine, which performs the steps of separating the body and the cap, filling the body with the fill and rejoining the body and the cap in the final-locked state.

The capsule filling machine used can be a capsule filling machine, preferably a fully automated capsule filling machine, that is capable to produce filled and closed capsules at a speed with an output of 1,000 or more filled and finally closed capsules per hour. Capsule filling machines, preferably fully automated capsule filling machines, are well known in the art and commercially available from several companies.

The capsule filling machine used can be preferably operated at a speed with an output of 1,000 or more, preferably 10,000 or more, 100,000 or more, 10,000 up to 500,000, filled and finally closed capsules per hour.

### Capsule Filling Machine General Operations

Before the capsule filling process, the capsule filling machine is provided with a sufficient number or amount of pre-coated hard-shell capsules in the pre-locked state. The capsule filling machine is also provided with enough amounts of fill to be filled in during operation.

The hard-shell capsules in the pre-locked state may fall by gravity into feeding tubes or chutes. The capsules can be uniformly aligned by mechanically gauging the diameter differences between the cap and the body. The hard-shell capsules are then usually fed, in proper orientation, into a two-section housing or brushing. The diameter of the upper bushing or housing is usually larger than the diameter of the capsule body bushing; thus, the capsule cap can be retained within an upper bushing while the body is pulled into a lower bushing by vacuum. Once the capsule is opened/the body and the cap are separated, the upper and lower housing or bushing are separated to position the capsule body for filling.

The open capsule body is then filled with the fill. Various types of filling mechanisms can be applied, with respect to the different fillings such as granules, powders, pellets or mini-tablets. Capsule filling machines in general employ a variety of mechanisms to handle the various dosage ingredients as well as various numbers of filling stations. The dosing systems are usually based on volumetric or amounts of fills governed by the capsule size and capacity of the capsule body. The empty capsule manufacturers usually provide reference tables that indicate the volume capacity of their capsule body and the maximum fill weight for different capsule sizes based on the density of the fill material. After the filling, the body and the cap are rejoined by the machine in the final-locked state or position.

### Use/Method of Use/Method Steps

The process for preparing a polymer-coated hard-shell capsule suitable as described herein can be understood as a method of use of a hard-shell capsule comprising a body and a cap, wherein in the closed state the cap overlaps the body either in a pre-locked state or in a final-locked state, for preparing a polymer-coated hard-shell capsule, suitable as container for pharmaceutical or nutraceutical biologically active ingredients, comprising the steps of
a) providing the hard-shell capsule is provided in the pre-locked state and
b) spray-coating with a coating solution, suspension or dispersion comprising a polymer or a mixture of polymers to create a coating layer which covers the outer surface of the hard-shell capsule in the pre-locked state.

The spray-coating can be preferably applied by using a drum coater equipment or a fluidized bed coating equipment. A suitable product temperature during the spray-coating process can be in the range from about 15 to 40, preferably from about 20 to 35° C. A suitable spray rate can be in the range from about 0.3 to 17.0, preferably 0.5 to 14 [g/min/kg]. After spray-coating a drying step is included.

The polymer-coated hard-shell capsule in the pre-locked state can be opened in a step c), filled with a fill comprising a pharmaceutical or a nutraceutical biologically active ingredient in a step d), and is then closed in a step e) to the final-locked state.

Steps c) to e) can be performed manually or preferably supported by a suitable equipment, for instance a capsule-filling machine. Preferably, the coated hard-shell capsule in the pre-locked state is provided to a capsule-filling machine, which performs the opening step c), the filling with a fill comprising a pharmaceutical or a nutraceutical biologically active ingredient in step d) and the closing of the capsule to the final-locked state in step e).

The selection of the processes in all their generic or specific features and embodiments as disclosed herein can be combined without restriction with any other generic or specific selections of materials or numerical features and embodiments as disclosed herein, such as polymers, capsule materials, capsule sizes, coating thicknesses, biologically active ingredients and any other embodiments as disclosed.

### Preparation of a Pharmaceutical or Nutraceutical Dosage Form

Disclosed is a process for preparing a pharmaceutical or nutraceutical dosage form comprising a polymer-coated hard-shell capsule in the final-locked state containing a fill comprising at least one pharmaceutical or nutraceutical biologically active ingredient, wherein the polymer-coated hard-shell capsule comprises a coating layer comprising a polymer or a mixture of polymers, where the coating layer covers the outer surface area of the capsule in the pre-locked state. Since the outer surface area of the capsule in the pre-locked state is larger than outer surface area of the capsule in the final-locked state a part of the polymer coating layer is hidden or enclosed between the body and the cap of the hard-shell capsule, which provides an efficient sealing.

Also disclosed is a method for protecting a pharmaceutically or nutraceutically active ingredient from atmospheric oxygen in a pharmaceutical delivering system comprising a hard-shell capsule and a fill, the fill comprising at least one pharmaceutically or nutraceutically active ingredient, wherein the precoated hard-shell capsule according to the present invention as described herein is provided in a pre-locked state to a capsule-filling machine, which performs the steps of separating the body and the cap, filling the body with the fill and re-joining the body and the cap in the final-locked state.

### Items

1. A precoated hard-shell capsule with at least one coating layer, suitable as container for pharmaceutical or nutraceutical biologically active ingredients, wherein the at least one coating layer comprises
i) at least one (meth)acrylate copolymer with a weight average molar mass from 25,000 to 900,000 g/mol and having a glass transition temperature (T_{gm}) from 0 to 200° C., wherein the (meth)acrylate copolymer comprises maximally 50% by weight of methacrylic acid monomer units,
ii) at least one plasticizer with a solubility in water of at least 50 g/l in an amount of maximally 50% by weight relative to the amount of the (meth)acrylate copolymer,
iii) at least one emulsifier with a melting point higher than 40° C. and having a hydrophilic/lipophilic balance (HLB) of 3 to 6,
iv) at least one emulsifier with a melting point lower than 40° C. and having a hydrophilic/lipophilic balance (HLB) of 8 to 16,
and wherein the at least one coating layer has an oxygen permeability of at most 2,500 ml/(m²·d·atm) measured at a thickness of the least one coating layer of 100 μm.
2. The precoated hard-shell capsule of item 1, wherein the at least one emulsifier with a melting point higher than 40° C. and having a hydrophilic/lipophilic balance (HLB) of 3 to 6 is present in an amount of maximally 25% by weight relative to the amount of the (meth)acrylate copolymer and wherein and the at least one emulsifier with a melting point lower than 40° C. and having a hydrophilic/lipophilic balance (HLB) of 8 to 16 is present in an amount of maximally 10% by weight relative to the amount of the (meth)acrylate copolymer.
3. The precoated hard-shell capsule of items 1 or 2, wherein the base material of the hard-shell capsule is selected from hydroxypropyl methyl cellulose, starch, gelatine, pullulan and a copolymer of a C₁- to C₄-alkylester of (meth)acrylic acid and (meth)acrylic acid.
4. The precoated hard-shell capsule of any of the preceding items, wherein the at least one coating layer further comprises at least one glidant which is present in an amount of 3 to 75% by weight, based on the total weight of the at least one polymer
5. The precoated hard-shell capsule of item 4, wherein the at least one glidant is selected from silica, ground silica, fumed silica, kaolin calcium silicate, magnesium silicate, colloidal silicone dioxide, talc, stearate salts, sodium stearyl fumarate, starch and stearic acid or mixtures thereof.
6. The precoated hard-shell capsule of any of the preceding items, wherein the at least one (meth)acrylate copolymer has a glass transition temperature (T_{gm}) from 40 to 160° C.
7. The precoated hard-shell capsule of any of the preceding items, wherein the at least one plasticizer is present in an amount of 2 to 40% by weight, based on the total weight of the at least one (meth)acrylate copolymer.
8. The precoated hard-shell capsule of any of the preceding items, wherein the at least one plasticizer is selected from alkyl citrates, diethyl sebacate, polyethylene glycols, and propylene glycols or combinations thereof.
9. The precoated hard-shell capsule of any of the preceding items, wherein the at least one coating layer is present in an amount of 0.1 to 5.8 mg/cm².
10. The precoated hard-shell capsule of any of the preceding items, wherein the precoated hard-shell capsule comprises a body and a cap, wherein in the closed state the cap overlaps the body either in a pre-locked state or in a final-locked state.
11. The precoated hard-shell capsule of any of the preceding items, wherein the at least one coating layer has glass transition temperature T_{gm}≤45° C. as determined by Differential Scanning calorimetry (DSC) according to ISO 11357-2:2013-05.
12. The precoated hard-shell capsule according to item 11, wherein the at least one coating layer has glass transition temperature T_{gm} in the range of −60° C. to 45° C. as determined by Differential Scanning calorimetry (DSC) according to ISO 11357-2:2013-05, more preferably glass transition temperature T_{gm} in the range of −60° C. to 40° C. as determined by Differential Scanning calorimetry (DSC) according to ISO 11357-2:2013-05.
13. A method for preparing a pharmaceutical delivering system comprising a hard-shell capsule and a fill, the fill comprising at least one pharmaceutically or nutraceutically active ingredient, wherein the precoated hard-shell capsule according to any of the preceding claims is provided in a pre-locked state to a capsule-filling machine, which performs the steps of separating the body and the cap, filling the body with the fill and re-joining the body and the cap in the final-locked state.
14. A method for protecting a pharmaceutically or nutraceutically active ingredient from atmospheric oxygen in a pharmaceutical delivering system comprising a hard-shell capsule and a fill, the fill comprising at least one pharmaceutically or nutraceutically active ingredient, wherein the precoated hard-shell capsule according to any of the preceding claims is provided in a pre-locked state to a capsule-filling machine, which performs the steps of separating the body and the cap, filling the body with the fill and re-joining the body and the cap in the final-locked state.
15. A precoated hard-shell capsule with at least one coating layer, suitable as container for pharmaceutical or nutraceutical biologically active ingredients, wherein the at least one coating layer comprises
i) at least one (meth)acrylate copolymer with a weight average molar mass from 25,000 to 900,000 g/mol and having a glass transition temperature (T_{gm}) from 0 to 200° C., wherein the (meth)acrylate copolymer comprises maximally 50% by weight of methacrylic acid monomer units,
ii) at least one plasticizer with a solubility in water of at least 50 g/l in an amount of maximally 50% by weight relative to the amount of the (meth)acrylate copolymer,
iii) at least one emulsifier with a melting point higher than 40° C. and having a hydrophilic/lipophilic balance (HLB) of 3 to 6,
iv) at least one emulsifier with a melting point lower than 40° C. and having a hydrophilic/lipophilic balance (HLB) of 8 to 16, and wherein the glass transition temperature T_{gm} $45° C. as determined by Differential Scanning calorimetry (DSC) according to ISO 11357-2:2013-05, more preferably the glass transition temperature T_{gm} in the range of −60° C. to 45° C. as determined by Differential Scanning calorimetry (DSC) according to ISO 11357-2:2013-05, most preferably the glass transition temperature T_{gm} in the range of −60° C. to 40° C. as determined by Differential Scanning calorimetry (DSC) according to ISO 11357-2:2013-05.

### EXAMPLES

### Comparative Example: Oxygen Permeability of HPMC Film

### Film Preparation:

Methocel™ VLV was slowly added to water under continuous stirring for preparation of a 20% w/w aqueous solution. The film was prepared by casting using a 500 μm squeegee (four-sided applicator frame) and a Teflon® coated metal plate. The film with a size of 30×6 cm was dried at room temperature for 24 h. The film thickness after drying was approximately 150 μm.

Oxygen permeability measurement: On the film, the oxygen permeability was measured with the apparatus shown in FIG. 2. In an oxygenated atmosphere, the apparatus was filled and closed by screwing the two Creanit® plates. In normal atmosphere, the penetrating oxygen/time was measured by means of a PSt9 sensor. This sensor is commonly used for measurement of gaseous oxygen traces (offered e.g. by the company PreSens Precision Sensing GmbH, Germany).

| | Oxygen permeation normalized to |
|---|---|
| | 100 μm film thickness |
| | ml/(m² · d · atm) |
| HPMC film | 2567 |

### Inventive Example: Formulation 1

### Solution and Film Preparation

Water and Polysorbate 80 were heated to 72° C. under continuous stirring. Glycerol monostearate (GMS, 40-55%, type II) was added and the mixture was stirred for 30 min at 72° C. After cooling down to 50° C. or below triethyl citrate (TEC) was added under continued stirring. After cooling to room temperature, the resulting suspension was added to aqueous polymer dispersion A (30% solid substance, weight average molar mass of about 450,000 g/mol, glass transition temperature (T_{gm}): about 28° C.). After additional 30 min stirring the resulting dispersion was passed through a 260 μm sieve.

The film was prepared by casting using a 500 μm squeegee (four-sided applicator frame) and a Teflon® coated metal plate. The film with a size of 30×6 cm was dried at room temperature for 24 h. The film thickness after drying was approximately 150 μm.

Oxygen permeability measurement: On the film, the oxygen permeability was measured with the apparatus shown in FIG. 2.

### Comparative Example: Formulation 2

### Solution and Film Preparation

To aqueous polymer dispersion A (30% solid substance, same as for Formulation 1 used) water and dibutyl sebacate was added. After 30 min stirring the dispersion was passed through a 260 μm sieve.

The film was prepared by casting using a 500 μm squeegee (four-sided applicator frame) and a Teflon® coated metal plate. The film with a size of 30×6 cm was dried at room temperature for 24 h. The film thickness after drying was approximately 150 μm.

Oxygen permeability measurement: On the film, the oxygen permeability was measured with the apparatus shown in FIG. 2.

### Formulation Compositions:

| | | Formulation 1 | | Formulation 2 |
|---|---|---|---|---|
| | | Solid | | Solid |
| | Formulation 1 | Composition | Formulation 2 | Composition |
| Material | Composition | Percentage | Composition | Percentage |
| Polymer Dispersion | 30 g dry | 96.52% | 30 g dry | 97.1% |
| A** (30% aqueous | polymer | | polymer | |
| dispersion) | substance | | substance | |
| Triethyl citrate | 1.5% on ds* | 1.45% | — | — |
| (Plasticizer) | | | | |
| Dibutyl sebacate | — | — | 3.0% on ds* | 2.9% |
| Glycerol | 1.5% on ds* | 1.45% | — | — |
| monostearate | | | | |
| (GMS) (100%) | | | | |
| (Emulsifier, HLB ca | | | | |
| 3, 8, melting point | | | | |
| 62° C.) | | | | |
| Polysorbate 80 | 0.6% on ds* | 0.58% | — | — |
| (Emulsifier, HLB ca | | | | |
| 12, melting point | | | | |
| 21° C.) | | | | |
| Demineralized | 181% on ds* | n.a. | 179% on ds* | n.a. |
| Water*** | | | | |
| Solid content | 20% w/w | | 20% w/w | |

*Quantity based on dry polymer substance [%]
**Polymer Dispersion A: Core-Shell Polymer consisting of 75% ethyl acrylate and methyl methacrylate (2:1) and 25% methyl methacrylate and methacrylic acid (1:1), commercially available under the trade name EUDRAGIT ® FL 30 D-55
***Water added in addition to the water contained in the 30% aqueous polymer dispersion A

### Oxygen Permeation Results of Obtained Films:

| | Oxygen permeation normalized to |
|---|---|
| | 100 μm film thickness |
| | ml/(m² · d · atm) |
| Formulation 1 (inventive) | 550 |
| Formulation 2 (comparative) | 35938 |

The glass transition temperature of the coating composition was determined using Differential Scanning calorimetry (DSC) according to ISO 11357-2:2013-05. The result is tabulated below.

| | T_{g} |
|---|---|
| Formulation 1 (inventive) | −19.33° C. |
| Comparative (comparative example 7 | 52.33° C. |
| of WO2020/229178 A) | |

1. A precoated hard-shell capsule with at least one coating layer, suitable as container for pharmaceutical or nutraceutical biologically active ingredients,
wherein the at least one coating layer comprises
i) at least one (meth)acrylate copolymer with a weight average molar mass from 25,000 to 900,000 g/mol and having a glass transition temperature (T_{gm}) from 0 to 200° C., wherein the (meth)acrylate copolymer comprises maximally 50% by weight of methacrylic acid monomer units,
ii) at least one plasticizer with a solubility in water of at least 50 g/l in an amount of maximally 50% by weight relative to the amount of the (meth)acrylate copolymer,
iii) at least one emulsifier with a melting point higher than 40° C. and having a hydrophilic/lipophilic balance (HLB) of 3 to 6,
iv) at least one emulsifier with a melting point lower than 40° C. and having a hydrophilic/lipophilic balance (HLB) of 8 to 16,
and wherein the at least one coating layer has an oxygen permeability of at most 2,500 ml/(m²·d·atm) measured at a thickness of the least one coating layer of 100 μm.

2. The precoated hard-shell capsule of claim 1, wherein the at least one emulsifier with a melting point higher than 40° C. and having a hydrophilic/lipophilic balance (HLB) of 3 to 6 is present in an amount of maximally 25% by weight relative to the amount of the (meth)acrylate copolymer and wherein and the at least one emulsifier with a melting point lower than 40° C. and having a hydrophilic/lipophilic balance (HLB) of 8 to 16 is present in an amount of maximally 10% by weight relative to the amount of the (meth)acrylate copolymer.

3. The precoated hard-shell capsule of claim 1, wherein the base material of the hard-shell capsule is selected from hydroxypropyl methyl cellulose, starch, gelatine, pullulan and a copolymer of a C₁- to C₄-alkylester of (meth)acrylic acid and (meth)acrylic acid.

4. The precoated hard-shell capsule of claim 1, wherein the at least one coating layer further comprises at least one glidant which is present in an amount of 3 to 75% by weight, based on the total weight of the at least one polymer

5. The precoated hard-shell capsule of claim 4, wherein the at least one glidant is selected from silica, ground silica, fumed silica, kaolin calcium silicate, magnesium silicate, colloidal silicone dioxide, talc, stearate salts, sodium stearyl fumarate, starch and stearic acid or mixtures thereof.

6. The precoated hard-shell capsule of claim 1, wherein the at least one (meth)acrylate copolymer has a glass transition temperature (T_{gm}) from 40 to 160° C.

7. The precoated hard-shell capsule of claim 1, wherein the at least one plasticizer is present in an amount of 2 to 40% by weight, based on the total weight of the at least one (meth)acrylate copolymer.

8. The precoated hard-shell capsule of claim 1, wherein the at least one plasticizer is selected from alkyl citrates, diethyl sebacate, polyethylene glycols, and propylene glycols or combinations thereof.

9. The precoated hard-shell capsule of claim 1, wherein the at least one coating layer is present in an amount of 0.1 to 5.8 mg/cm².

10. The precoated hard-shell capsule of claim 1, wherein the precoated hard-shell capsule comprises a body and a cap, wherein in the closed state the cap overlaps the body either in a pre-locked state or in a final-locked state.

11. The precoated hard-shell capsule of claim 1, wherein the at least one coating layer has glass transition temperature T_{gm}≤45° C. as determined by Differential Scanning calorimetry (DSC) according to ISO 11357-2:2013-05.

12. The precoated hard-shell capsule according to claim 11, wherein the at least one coating layer has glass transition temperature T_{gm} in the range of −60° C. to 45° C. as determined by Differential Scanning calorimetry (DSC) according to ISO 11357-2:2013-05.

13. A method for preparing a pharmaceutical delivering system comprising a hard-shell capsule and a fill, the fill comprising at least one pharmaceutically or nutraceutically active ingredient, wherein the precoated hard-shell capsule according to claim 1 is provided in a pre-locked state to a capsule-filling machine, which performs the steps of separating the body and the cap, filling the body with the fill and re-joining the body and the cap in the final-locked state.

14. A method for protecting a pharmaceutically or nutraceutically active ingredient from atmospheric oxygen in a pharmaceutical delivering system comprising a hard-shell capsule and a fill, the fill comprising at least one pharmaceutically or nutraceutically active ingredient, wherein the precoated hard-shell capsule according to claim 1 is provided in a pre-locked state to a capsule-filling machine, which performs the steps of separating the body and the cap, filling the body with the fill and re-joining the body and the cap in the final-locked state.
